(19) United States
(12) Patent Application Publication
(10) Pub. No.: US 2012/0109305 A1
(43) Pub. Date: May 3, 2012

(54) Intervertebral cage having flexibility

(76) Inventor: Kyung-Woo Park, Seoul (KR)

(21) Appl. No.: 12/931,561

(22) Filed: Feb. 4, 2011

(30) Foreign Application Priority Data
Oct. 28, 2010 (KR) 10-2010-0106250

(51) Int. Cl. A61F 2/44

(52) U.S. Cl. 623/17.13

(57) ABSTRACT

An intervertebral cage having flexibility is provided wherein a housing itself has a plate spring form having proper elasticity. The housing can have a shape memory characteristic to obtain a modulus of elasticity suitable for differing spinal loads according to each patient, thereby absorbing a shock applied to a spine. A distance between disks is restored by the intervertebral cage to sufficiently secure a disc height, thereby relieving spinal nerve stress. The intervertebral cage may be converted from a simple fusion application into a functional cage adequate for a physiological biomechanics. The flexible intervertebral cage includes a housing having a closed sectional surface with an empty hollow therein. The housing itself has proper elasticity so that it absorbs a load by a stress applied in a vertical direction of a spine by a dynamic motion due to an upright walk of a patient to serve as a normal disk.

### CROSS-REFERENCE TO RELATED APPLICATIONS

This application claims priority under 35 U.S.C. §119 of Korean Patent Application No. 10-2010-0106250, filed on Oct. 28, 2010.

### BACKGROUND OF THE INVENTION

1. Field of the Invention

Exemplary embodiments of the present invention relate to an artificial disk used for disk treatment, and more particularly, to an intervertebral cage having flexibility, which absorbs a shock applied to the spine after a surgery and controls a movement of the spine to maintain a spinal sagittal balance and a sufficient disk height, thereby relieving stresses on spinal nerves.

2. Description of Related Art

Generally, healthy disks absorb a shock applied to the spine and restrict a movement of the spine to protect spinal nerves.

A disk disease that is one of the most common diseases includes a herniated lumbar disk in which a disk protrudes due to a serious shock applied to a waist and a degenerative disk in which an intervertebral disk between vertebral bodies is worn due to an aging thereof to stress peripheral nerve tissues.

In spinal diseases, when a degenerative change (aging phenomenon) of a disk is serious, natural functions of the disk become gradually lost. Thus, the disk may be vulnerable to physical shocks to cause pain. In addition, the degenerative change of the disk acts as a factor of an unstable spinal motion to stress the nerve tissues, thereby worsening pain.

The herniated lumbar disk of the disk diseases may be treated by existing disk surgery. However, it has been difficult to treat the degenerative disk until now. This is because a large number of patients undergoing degenerative disk suffer from adult diseases such as diabetes, hypertension, heart diseases, etc. at the same time. However, examples with respect to clinical trials of a recently developed spinal fusion technology are presented to break new ground in the treatment of the degenerative disk in which treatment is difficult.

When surgeons operate on patients suffering from degenerative spine diseases or spinal instability, an abnormal load transmission pattern may occur in cases wherein only a posterior dynamic stabilization device is used. As a result, most of the stress from motion is focused on the posterior dynamic stabilization device. This is the most critical failure factor with respect to a spinal surgery. Specifically, when a posterior dynamic stabilization system is constituted by rigid rods, the rods causes a stress shielding effect to have an abnormal load transmission pattern of the spines. As a result, when the spinal surgery is performed, it is necessary to use a cage for anterior stabilization.

Spinal fusion technology for treating spine diseases is the most advanced technology, and was developed in 1992 in the U.S.A. and has been approved in safety and effectiveness by the U.S. Food and Drug Administration (FDA). Also, the spinal fusion technology is widely performed in Korea.

Spinal fusion technology is a technology in which a cage formed of a harmless material such as titanium and peek is inserted between vertebral bodies with spinal diseases to secure a space, thereby relieving back pain. That is, an intervertebral disk, which does not perform its full functions between the vertebral bodies due to degeneration is removed to graft a harmless artificial disk such as T.F.C. (Threaded Fusion Cage) having a cylindrical shape into the position at which the intervertebral disk is removed.

Spinal fusion technology has been used for bone fusion. For this, a degenerative disk is removed, and a cage is inserted into the position at which the disk is removed to secure a space and graft bones around the cage, thereby fusing the bone. However, another limitation such as the restriction of spinal motion and the abnormal load transmission pattern may occur after fusion, so that a degenerative change of an adjacent segment may be promoted.

Since individual spinal conditions are different according to the age of person, an adequate cage for an artificial disk should be used when a disk is treated. However, the cage for an artificial disk does not have a differentiated structure applied to various spinal conditions of patients. Thus, only a cage having an adequate size was selected and grafted in all cases. As a result, this causes a fundamental limitation that a surgery which is optimal to patients is difficult.

Also, since the related art cage for an artificial disk requires various surgical instruments for graft operation, it is difficult to smoothly perform the graft operation. In addition, since large and various surgical instruments are used, it may have a bad influence on the nerve tissues of the spine during the operation. Also, it may take a long time to perform the graft operation.

To solve the above-described limitations, a variable artificial disk is disclosed in Korean Patent Publication No. 10-2004-0064577. As shown in FIG. 1, the variable artificial disk includes a boss part 103 for supporting a cylindrical frame and a housing 102 including an independent plate 104 coupled to the boss part 103 and expanded in a radial direction. A male screw is disposed within a slit 106 defined in the independent plate 104. When the male screw is rotated, the independent plate 104 is expanded in the radial direction. Due to such a structure, when a height difference between a front end and a rear end of the grafted artificial disk is needed to maintain an adequate bending state of a patient's spine, the artificial disk may be grafted while a distance between the front and rear ends is adequately adjusted. Thus, an adequate treatment may be possible according to the conditions of the patients.

However, although a disc height can be adjusted through the above-described structure, the variable artificial disk is used for spinal fusion. Thus, since a spinal motion is restricted, there is a limitation that variable artificial disk does not take against the degenerative change.

Also, a prosthetic instrument for a replacing spine disk is disclosed in U.S. Pat. No. 6,964,686. As shown in FIG. 2, in the prosthetic instrument for replacing the spine disk, a slit 206 having a spring shape and function is defined in a circumference of a housing 202 having an axially elongated hollow 204. A lower disk support 208 having a concave shape and an upper disk support 210 in which a groove for receiving the concave shape of the lower disk support 208 is defined are inserted into the axially elongated hollow 204. Such a structure serves as a structure, which is buffered by the slit 206 of the housing 202 about the lower disk support 208 as vertebral bodies press the upper disk support.

Although the structure can perform the buffering function by the slit 206 of the housing 202, the structure does not secure a sufficient disk distance. In addition, since the structure is buffered only a vertical direction, it is impossible to control the vertebral bodies so that they are moved in a free direction. Also, there is a limit to execution of a natural function of the disk maintaining a spinal sagittal balance.

Alternatively, block cages formed of a titanium alloy named as Ti6Al4V or a synthetic resin of polyetheretherketone (Peek) are being proposed as typical cages for fusion, which are known up to now.

However, the cages may be buried into the vertebral body by a motion effect of the patient after the surgery. As well known, an elastic modulus of Peek is greater than that of Ti6Al4V and similar to that of a vertebral end-plate. The Peek material block cage may be further preferred to the Ti6Al4V block cage because a rate in which the Peek material block cage is buried into the vertebral body is lower than that in which the Ti6Al4V block cage is buried into the vertebral body. A rate at which the Peek material block cage is buried into the vertebral body is about 20% to about 30%. Also, a rate at which the Ti6Al4V block cage is buried into the vertebral body is about 40% to about 60%. Thus, the Peek material block cage may be superior to the Ti₆Al₄V block cage. However, in case of patients suffering from osteopenia or osteoporosis, when a surgeon uses the block cage formed of the Peek material, there is a limitation that the cage may be buried within several years after the surgery.

### SUMMARY OF THE INVENTION

An embodiment of the present invention is directed to an intervertebral cage having flexibility in which a usage object of the intervertebral cage may be converted from a simple fusion into a functional cage adequate for a physiological biomechanics.

Another embodiment of the present invention is directed to an intervertebral cage having flexibility in which a housing itself has a plate spring form having proper elasticity, a shape memory characteristic is granted to the housing to obtain moduli of elasticity accessible to spinal loads which differ according to each patient, thereby absorbing a shock applied to a spine, and a distance between disks is restored to sufficiently secure a disc height, thereby relieving spinal nerve stress.

Another embodiment of the present invention is directed to an intervertebral cage having flexibility in which a surgery can be easily and adequately done in a narrow disk space.

Another embodiment of the present invention is directed to an intervertebral cage having flexibility, in which a housing itself is elastically moved to control a free spinal motion, thereby maintaining a spinal sagittal balance.

Another embodiment of the present invention is directed to an intervertebral cage having flexibility, which can recover somewhat physiologically adequate functions of a normal disk from a damaged disk in which a motion is stopped due to a related art complete fusion, a sagittal balance is broken, and proper functions of the disk are damaged.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

In accordance with an embodiment of the present invention, an intervertebral cage having flexibility includes: a housing having a closed sectional surface with an empty hollow therein, wherein the housing itself has proper elasticity so that it absorbs a load by a stress applied in a vertical direction of a spine by a dynamic motion due to an upright walk of a patient to serve as a normal disk.

The housing may include a first elastic part vertically buffered by an inner space thereof; and a second elastic part bent so that it is inserted into the hollow from a side surface of the first elastic part, the second elastic part being configured to provide a buffering force together with the first elastic part.

The first and second elastic parts may include an oval-shaped plate spring having a substantially U- or W-shaped closed sectional surface.

The housing may be formed from one or both of a titanium alloy and a nitinol alloy, and a shape memory characteristic may be granted to the first and second elastic parts of the housing.

In accordance with another embodiment of the present invention, an intervertebral cage having flexibility includes: a housing including an oval-shaped plate having a hollow section, which is empty therein, the housing having an opening with a side opened and executing proper elasticity itself; and a clip plate inserted into an inner surface of the hollow through the opening of the housing, the clip plate being buffered by being cooperated with buffering of the housing.

One of the housing and the clip plate may be formed of a titanium alloy or a nitinol metal and the other one of the housing and the chip plate may be formed of a titanium alloy.

### BRIEF DESCRIPTION OF THE DRAWINGS

FIGS. 1 and 2 are views of a related art artificial disk.

FIG. 3 is a sectional view of an intervertebral cage having flexibility in accordance with an embodiment of the present invention.

FIG. 4 is a view illustrating a modified example of FIG. 3.

FIG. 5 is a sectional view of an intervertebral cage having flexibility in accordance with another embodiment of the present invention.

FIG. 6 is a view illustrating a modified example of FIG. 5.

FIG. 7 is a front-sectional view of an intervertebral cage having flexibility in accordance with another embodiment of the present invention;

FIG. 8A is a side cross-sectional view showing the first step of a process for inserting a housing inside of a body;

FIG. 8B is a side cross-sectional view showing the second step of a process for inserting a housing inside of a body; and

FIG. 8C is a side cross-sectional view showing a third step of a process for inserting a housing inside of a body.

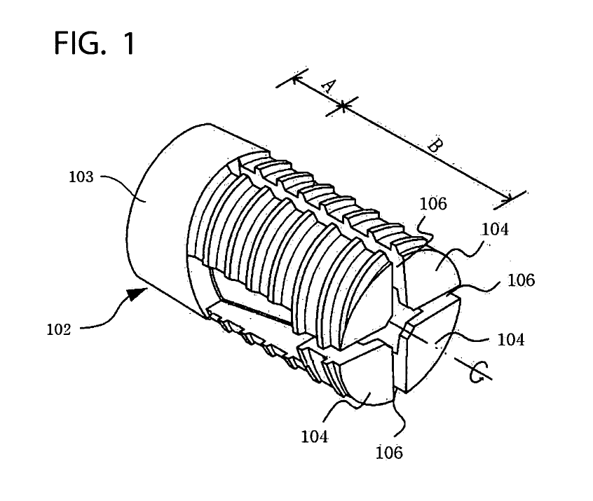

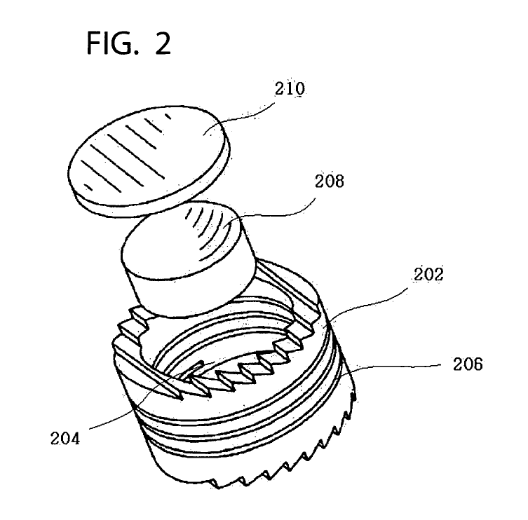

### DETAILED DESCRIPTION OF THE DRAWINGS

Objects, other objects, characteristics and advantages of the present invention will be easily understood from an explanation of a preferred embodiment that will be described in detail below by reference to the attached drawings.

An intervertebral cage having flexibility in accordance with the present invention may absorb a shock applied to a spine and secure a distance between disks to control a spinal motion. Thus, the intervertebral cage may serve as a normal disk.

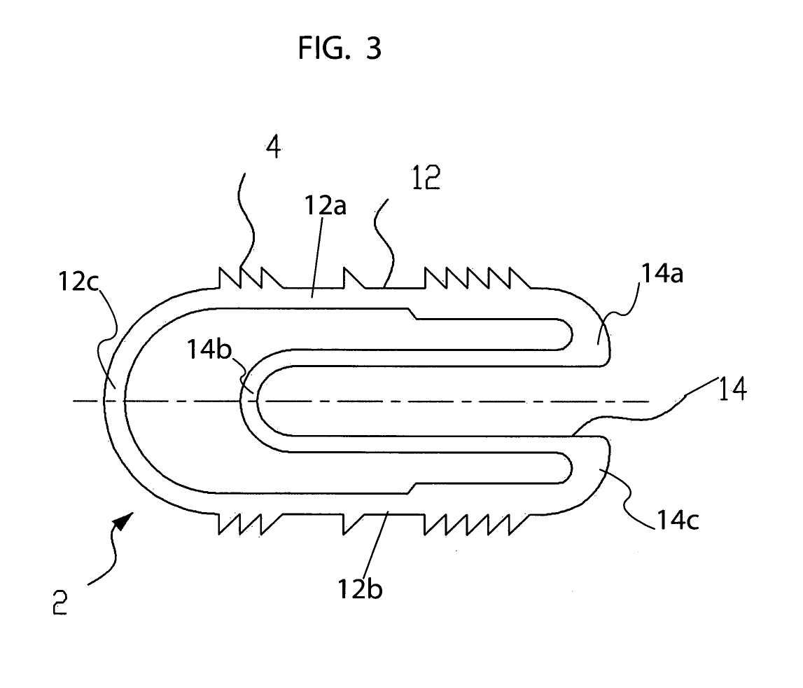

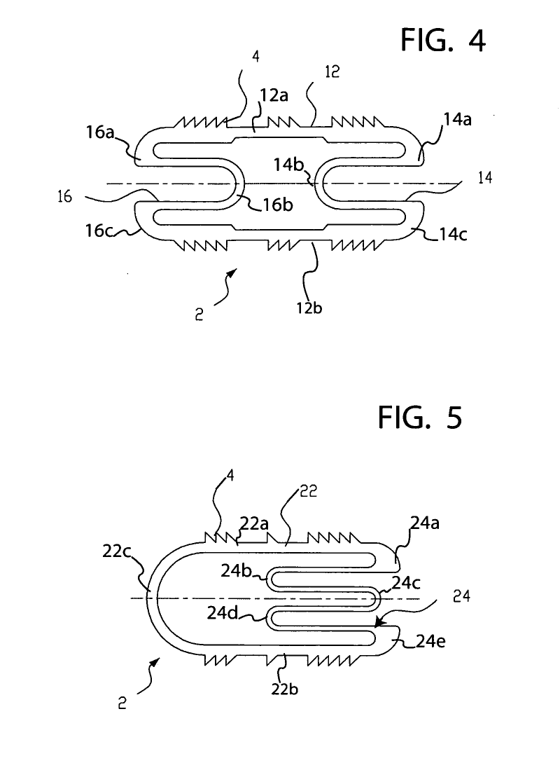

FIG. 3 is a sectional view of an intervertebral cage having flexibility in accordance with an embodiment of the present invention, and FIG. 4 is a view illustrating a modified example of FIG. 3.

The embodiments in accordance with the present invention may have a structure adequately applicable to lumbar or cervical vertebrae.

As shown in FIG. 3, the cage according to an embodiment includes a housing 2 having a hollow section, which is empty therein and executing proper elasticity itself and a plurality of protrusions 4 disposed on outer upper and lower surfaces of the housing 2 and closely fused with a vertebral body. The protrusions 4 may have a toothed shape on top and bottom surfaces of the housing 2 through a knurling process.

Preferably, the housing 2 includes a first elastic part 12 vertically buffered by an inner space and a second elastic part 14 bent so that it is inserted into the hollow from a side surface of the first elastic part 12 and providing a buffering force together with the first elastic part 12. Also, a certain portion of a section connected from the first elastic part 12 to the second elastic part 14 has a thickness greater than that of the second elastic part 14 to prevent the buffering force of the second elastic part 14 from being reduced, thereby increasing durability.

The first and second elastic parts 12 and 14 of the housing 2 have a plate spring structure having a substantially U-shaped closed sectional surface. Thus, a load acting in vertical direction of the spine may be absorbed by an inner space of the first elastic part 12 and a U-shaped space of the second elastic part 14.

The housing 2 may provide a strong supporting force by the plate spring structure. In addition, the buffering effect of the housing 2 may provide good stability (or fusion characteristic) at a side between a vertebral end-plate and a cage surface by a Wolff's law.

Also, the housing 2 vertically executes the buffer force by the inner space to form a physiologically good load transfer (or distribution) pattern without having a stress shielding effect generated when a load is applied to the spine. Thus, the housing 2 absorbs a load by a stress applied in a vertical direction of the spine by a dynamic motion due to an upright walk of a patient, i.e., from an upstream spine to a downstream spine to serve as a normal disk.

In case of a block cage formed of a titanium material (for example, alloy No. Ti6Al4V) or polyetheretherketone (Peek) that is a synthetic resin, which is indicated as an existing limitation, the block cage may be buried into a vertebral body after a surgery. However, the structure of the housing 2 in accordance with the present invention may prevent the cage from being buried into the vertebral body through a shock absorption mechanism.

More particularly, housing 2 has at least a first elastic part or spring such as elastic part 12 which is formed as an outer portion of the housing and which can be formed substantially C-shaped joining first and second ends 12a and 12b, via an intermediate portion 12c. This housing, 2 based upon its design, and after it is inserted into a patient, is capable of absorbing stress applied to a person's spine based upon the dynamic motion of that person. This dynamic motion can include not only vertical motion, but twisting, bending, arching one's back or turning. This type of dynamic motion can result in non-linear stresses applied to a person's back such as through torque, or motion in at least two different directions.

The second elastic part 14 is formed inside of the first elastic part 12c, wherein this second elastic part can be substantially C-shaped. Both the first elastic part 12c and the second elastic part can function substantially as springs, functioning as a leaf or natural spring formed integral with the remainder of the housing (See FIG. 3). This housing such as that shown in FIG. 3 has a plurality of different spring like flexion points such as that formed by elastic part or spring 12c, or elastic parts 14a, 14b, and 14c which flex when encountering pressure or force from an adjacent element such as a body part. With this design, elastic parts 14a and 14c are thicker than elastic part 14b. Coupled to each end 12a and 12b are protrusions 4. These protrusions are configured to fuse with a vertebral body.

As a modified example of the current embodiment of the present invention, as shown in FIG. 4, a third elastic part 16 may be disposed on the other surface of the first elastic part 12 of the housing 2. The third elastic part 16 may have a shape symmetric to that of the second elastic part 14, i.e., a U shape bent so that it is inserted into from the other side surface of the first elastic part 12 to an inside surface. In the modified example, a load vertically applied to the housing 2 is primarily absorbed by the first elastic part 12 and secondarily absorbed by the second and third elastic parts 14 and 16.

A circular rod formed of a metal material digs therein so that a vertically elastic distance is set to about 1 mm to about 2 mm to manufacture the housing 2 having a hollow circular plate with a thickness of about 1.5 mm. Also, the housing may have a length of about 24 mm, a height of about 12 mm, and a width of about 1 mm so that it is smoothly inserted into the intervertebral.

The housing 2 including the above-described components has a proper elasticity itself such as an effect of a plate spring. Thus, the housing 2 is inserted into a portion in which a degenerative spine disease occurs or a portion in which spinal instability occurs to fuse the intervertebral and perform physiologically adequate functions. Specifically, the housing 2 elastically buffers and absorbs a shock vertically applied to the vertebral body to restrict a motion of the intervertebral.

In the embodiment of the present invention, the housing 2 may be formed of one or both of titanium or nitinol (Ni—Ti) alloys. Specifically, the housing 2 may have a structure having a shape memory characteristic in which a crystal structure is changed according to a change of temperature.

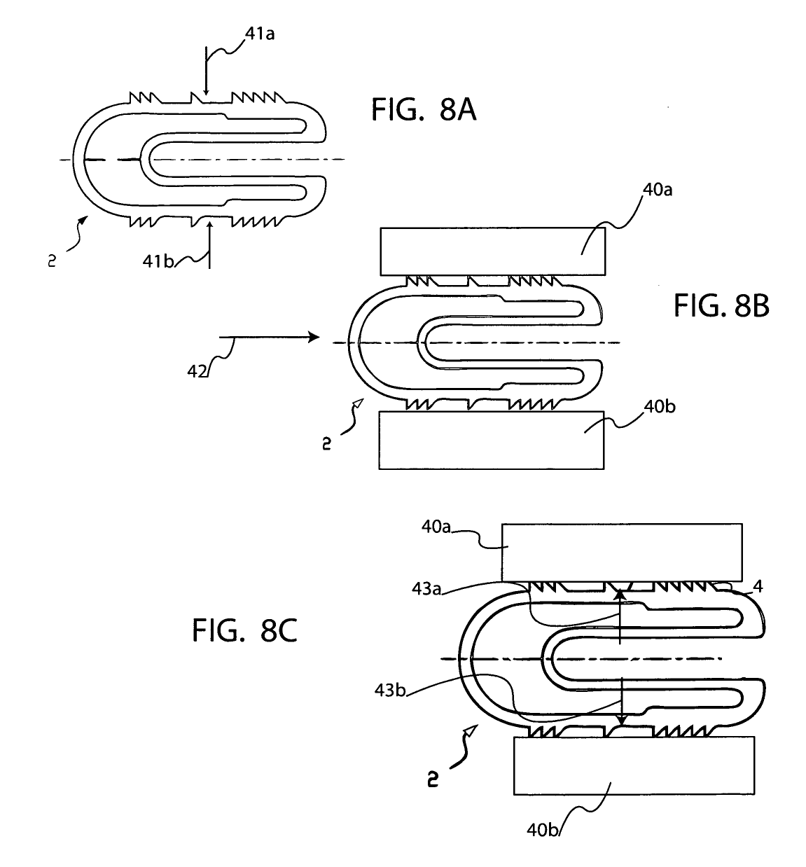

In more detail, the housing 2 can have a shape memory characteristic in which an elastic distance is beginning to close at a low temperature of about 4 degrees Celsius and returned to an original position at a temperature of about 28 degrees Celsius lower than a body temperature. (See FIGS. 8A-8C).

The cage may be easily inserted into the intervertebral using the material characteristic of the housing 2 during the surgery. That is, during the surgery, when the housing 2 is immersed into a cool solution (about 4 degrees Celsius) to narrow a vertical distance of the housing 2, a total height of the cage is reduced to allow the cage to be easily inserted into the intervertebral. After the surgery, the height of the housing 2 is restored by the body temperature to maintain a disk height by a distance between normal disks. The elastic operations of the first and second elastic parts 12 and 14 absorb the shock applied to the spine when the vertebral body is freely moved and restrict a motion of the vertebral body to maintain the spinal sagittal balance. Specifically, the housing 2 reconstructs a load transfer (distribution) pattern similar to that of the normal disk to execute good anterior stability without the help of an anterior support having a large volume. Also, it may prevent a posterior dynamic stabilization from being failed by an abnormal load transfer pattern.

Another embodiment of the present invention will be described with reference to FIGS. 5 and 6.

FIG. 5 is a sectional view of an intervertebral cage having flexibility in accordance with another embodiment of the present invention. FIG. 6 is a view illustrating a modified example of FIG. 5. The current embodiment has the same material and component as the foregoing embodiment. In detail, the current embodiment has the same component as the foregoing embodiment except that the first and second elastic parts 22 and 23 of a housing 2 have a W-shaped closed sectional surface. More particularly, shown in FIG. 5, there are a plurality of C-shaped natural or leaf springs which include spring 22c which is coupled at each end to end 22a or 22b. These springs or elastic elements comprise a first spring 22a, a second spring 24b, a third spring 24c, a fourth spring 24d, and a fifth spring 24e. These springs extend in a serpentine manner or accordion shaped manner to form multiple springs. Essentially, since these springs or elastic elements 24a, 24b, 24c, and 24d are stacked one on top of the other they form a first spring at a first end of the cage or body with a second spring being formed by section 22c which can be of a thicker dimension than spring or elastic element 24. Coupled to each end 12a and 12b are protrusions 4.

As shown in FIG. 6, a fourth elastic part 26 having a W-shape symmetric to that of the second elastic part 24 may be disposed on the other surface of the first elastic part 22 of the housing 2. More particularly, regarding FIG. 6, includes a body formed form an elastic material 22 which includes at least two sets of different springs 24 and 26 disposed on each side of the body. First spring 24 includes individual springs 24a, 24b, 24c, 24d and 24e which can be of any shape but in this case are shown C-shaped and which extend in a serpentine or accordion shaped manner from first end 22a to second end 22b. Coupled to these ends 22a and 22b are protrusions 4.

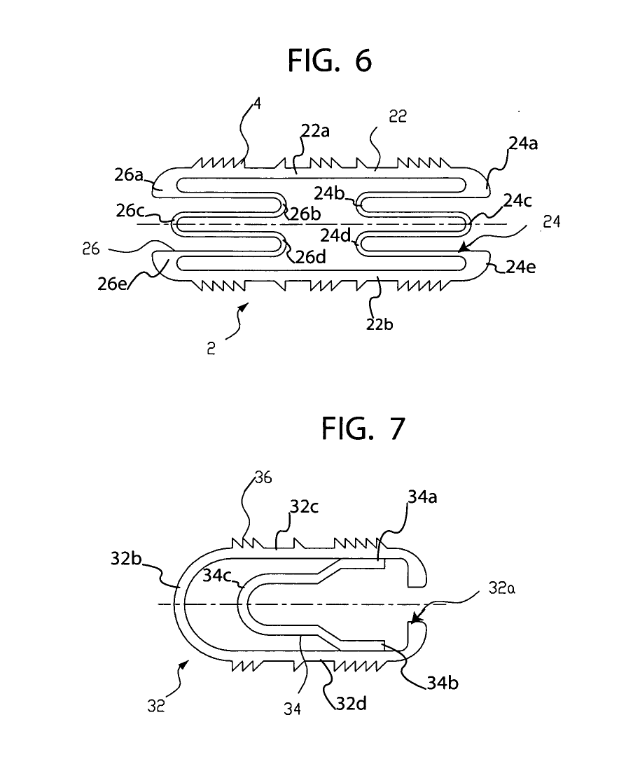

FIG. 7 is a front-sectional view of an intervertebral cage having flexibility in accordance with another embodiment of the present invention.

As shown in FIG. 7, a cage according to the current embodiment includes a housing 32 having a hollow section, which is empty therein and an opening 32a with a side opened and executing proper elasticity itself, a U-shaped clip plate 34 inserted into an inner surface of the hollow through the opening 32a of the housing 32 and buffered by being cooperated with buffering of the housing 32, and a plurality of protrusions 36 disposed on outer upper and lower surfaces of the housing 32 and closely fused with one or more vertebral bodies.

In accordance with the current embodiment, the housing 32 is buffered by a shock load applied to a top surface of the housing 32 to primarily absorb the shock load. Then, the clip plate 34 cooperated with the housing is buffered within a distance of the opening 32a to secondarily absorb the shock load.

In the current embodiment, one of the housing 32 and the clip plate 34 is formed of a titanium alloy or a nitinol metal, which has a shape memory characteristic, and the other one is formed of a titanium alloy.

In accordance with the exemplary embodiments of the present invention, the following functional effects and effects of surgery aspects may be provided. In the overall effects, when the load is applied to the vertebral body, the physiologically good load transfer (or distribution) pattern may be formed without having the stress shielding effect. Also, a buried rate of the cage into the vertebral body may be reduced to about 10% or less by the shock buffering mechanism. In addition, the good stability (or fusion characteristic) may be realized between the vertebral end-plate and the cage by the Wolff's law. Also, the strong supporting force may be executed by the characteristic of the plate spring of the case and the material characteristic of the nitinol alloy.

The above-described effects will be described below in more detail.

Firstly, since the housing having the closed sectional surface is configured to elastically act itself, the housing may be buffered against the vertical shock to absorb the shock. Also, the disk distance may be sufficiently secured by a distance corresponding to the normal disk through the elastic characteristic of the housing itself to release the spinal nerve stress.

Secondly, if the cage has a weak supporting force, the disk height is not maintained and a lateral foramen is not opened. However, since the cage has the durability and elasticity by the nitinol material and the plate spring structure, the disk height may be maintained.

Thirdly, since a shape memory characteristic may be granted to the housing of the cage, the cage can be compressed to minimize the disk height when the cage is inserted between vertebral bodies to allow the cage to be easily inserted between vertebral bodies through a narrow space of the patient's spine. Also, after the surgery, the distance between vertebral bodies is restored to the height of the cage to sufficiently secure a distance between vertebral bodies, thereby relieving spinal nerve stress. Specifically, since the cage is smoothly seated in position by the shape memory characteristic, a mis-positioning of the cage indicated as a limitation when the existing block cage is used may be prevented.

Fourthly, the spinal motion may be physiologically restricted by the elastic movement of the housing itself to maintain the spinal sagittal balance.

Fifthly, the objective of the intervertebral cage may be converted from the simple fusion into the functional case adequate for the physiological biomechanics to obtain improved clinical results.

One of the benefits of the designs of FIGS. 3-7 is that these housings or cages are capable of bending or flexing in multiple different directions so that a patient who receives this device would be able to bend or flex in nearly any direction with the device bending or flexing to compensate. This design, with the different elastic elements is not limited to simply alleviating vertical compressive forces between two vertebrae.

FIGS. 8A-8C show an illustration of a process for inserting a cage or housing into a person's body. This process includes first the shrinking of the expansion of the design of FIG. 3 as shown in FIG. 8A and by arrows 41a and 41b. This step can be accomplished by immersing this housing 2 in a low temperature bath of at or below 4 degrees Celsius. Next, after the housing is inserted in between two intervertebral bodies 40a and 40b, as shown by the direction of arrow 42, this housing heats up via the body temperature of the patient thereby expanding between these two bodies 40a and 40b as shown by arrows 43a and 43b. As shown protrusions 4 mesh with the vertebrae bone 40a and 40b to lock the housing or cage within a user's body. This same procedure can be accomplished using any one of the other embodiments shown in FIGS. 4-7.

As described above, the intervertebral cage having the elasticity may replace the normal disk through the foregoing effects.

While the present invention has been described with respect to the specific embodiments, it will be apparent to those skilled in the art that various changes and modifications may be made without departing from the spirit and scope of the invention as defined in the following claims.

What is claimed is:

1. An intervertebral cage having flexibility comprising: a housing having a closed sectional surface with an empty hollow therein,
wherein said housing is configured to be sufficiently elastic so that it absorbs a load by a stress applied in a vertical direction of a spine by a dynamic motion due to an upright walk of a patient to serve as a normal disk.

2. The intervertebral cage of claim 1, wherein said housing comprises at least one elastic part comprising a spring.

3. The intervertebral cage of claim 1, wherein said housing comprises a first elastic part having a hollow section vertically buffered by an inner space of said hollow section; and a second elastic part bent so that it is inserted into said hollow section from a side surface of said first elastic part, said second elastic part being configured to provide a buffering force together with said first elastic part.

4. The intervertebral cage of claim 3, wherein said first and second elastic parts comprise a plate spring having a parts that comprise substantially an oval-shaped U-shaped closed sectional surface.

5. The intervertebral cage of claim 3, wherein said first and second elastic parts comprise an oval-shaped substantially W-shaped closed sectional surface.

6. The intervertebral cage of claim 4, further comprising a third elastic part wherein said second elastic part is disposed on a first surface of said first elastic part and said third elastic part is disposed on a second surface of said first elastic part, said third elastic part having a shape symmetric to that of said second elastic part.

7. The intervertebral cage of claim 6, further comprising a fourth elastic part disposed on a third surface of said first elastic part, wherein said fourth elastic part having a shape symmetric to that of the second elastic part.

8. The intervertebral cage of claim 2, wherein said spring comprises at least a nitinol alloy.

9. The intervertebral cage of claim 1, wherein said housing is formed of at least one of a titanium alloy and a nitinol alloy.

10. The intervertebral cage of claim 3, wherein said first and second elastic parts of said housing have a shape memory characteristic.

11. The intervertebral cage of claim 3, wherein at least a portion of a section connected from said first elastic part to said second elastic part has a thickness greater than that of said second elastic part.

12. The intervertebral cage of claim 1, wherein said housing further comprises:
an upper plate having a top surface;
a first plurality of protrusions disposed on said top surface of said upper plate;
a lower plate having an under surface; and
a second plurality of protrusions disposed on said under surface of said lower plate;
wherein said first plurality of protrusions is configured to be closely fused to a first vertebral body and said second plurality of protrusions is configured to be closely fused to a second vertebral body.

13. An intervertebral cage having flexibility comprising:
a housing comprising a substantially oval-shaped plate having a hollow section, which is empty therein, said housing having an opening with a side opened and executing proper elasticity itself; and
a clip plate disposed in an inner surface of said hollow section through the opening of said housing, said clip plate acting as a spring in response to a movement of said housing.

14. The intervertebral cage of claim 13, wherein one of said housing and said clip plate is formed of a titanium alloy or a nitinol metal and the other one of said housing and said clip plate is formed of a titanium alloy.

15. The intervertebral cage of claim 1, wherein said housing comprises:
a) a first surface having a first set of protrusions;
b) a second oppositely spaced surface having a second set of protrusions facing opposite said first set of protrusions; and
c) at least one spring coupling said first surface to said second surface, wherein said spring is formed integral with said first surface and said second surface.

16. The intervertebral cage of claim 15, wherein said at least one spring comprises a C-shaped leaf spring coupling said first surface to said second oppositely spaced surface.

17. The intervertebral cage of claim 15, wherein said housing is substantially U-shaped and said at least one spring comprises at least one first spring that is substantially C-shaped and at least one second spring that is substantially C-shaped.

18. The intervertebral cage of claim 15, wherein said housing is substantially H-shaped and wherein said housing comprises at least two springs that are substantially C-shaped.

19. The intervertebral cage of claim 15, wherein said housing is substantially W-shaped and wherein said housing comprises at least three springs that are substantially C-shaped.

20. The intervertebral cage of claim 15, wherein said at least one spring comprises at least one U-shaped clip plate having a first end and a second end, wherein said housing has a first end and a second end, wherein said first end of said housing is formed by said first surface, and said second end of said housing is formed by said second surface, said housing further comprising a first inner surface coupled to said first end and spaced opposite said first surface, and a second inner surface coupled to said second end and spaced opposite said second oppositely spaced surface, wherein said first end of said U-shaped clip plate is coupled to said first inner surface, and said second end of said U-shaped clip plate is coupled to said second inner surface.

21. A method for inserting an intervertebral cage inside a body, the method comprising the steps of:
a) narrowing at least one dimension of a housing for an intervertebral cage in a temporary manner;
b) inserting said housing into a body adjacent to at least one vertebrae.

22. The method of claim 21, wherein said step of narrowing at least one dimension of a housing comprises cooling said housing to narrow the at least one dimension of the housing.

23. The method of claim 21, wherein said step of narrowing at least one dimension of a housing includes narrowing a vertical dimension of the housing, the vertical dimension being based upon an upright position of a person receiving the intervertebral cage.

24. The method of claim 21, further comprising the step of expanding said at least one dimension back to approximately its original dimension after the housing is inserted into a user's body.
